(19) United States
(12) Patent Application Publication
(10) Pub. No.: US 2006/0064002 A1
(43) Pub. Date: Mar. 23, 2006

(54) Method for monitoring thermal heating during magnetic resonance imaging

(76) Inventors: Thomas M. Grist, Madison, WI (US); Yong Zhou, Waukesha, WI (US)

Correspondence Address: QUARLES & BRADY LLP, 411 E. WISCONSIN AVENUE, SUITE 2040, MILWAUKEE, WI, 53202-4497, US

(21) Appl. No.: 10/945,377

(22) Filed: Sep. 20, 2004

(51) Int. Cl. A61B 5/05

(52) U.S. Cl. 600/410

(57) ABSTRACT

The SAR exposure of a subject undergoing an MRI examination is measured by acquiring a thermal image that indicates that temperature increase caused by the SAR exposure. This measurement may be used in a prescan process to adjust the SAR load produced by a prescribed imaging pulse sequence, and it can be used during the scan to adjust the SAR load produced by the prescribed imaging pulse sequence.

### STATEMENT REGARDING FEDERALLY SPONSORED RESEARCH

This invention was made with government support under Grant Nos. HL067029 and CA86278 awarded by the National Institute of Health. The United States Government has certain rights in this invention.

### BACKGROUND OF THE INVENTION

The field of the invention is nuclear magnetic resonance imaging (MRI) methods and systems. More particularly, the invention relates to the measurement and limitation of RF power produced by an MRI system during a patient scan.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a radio frequency (RF) magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited spins after the excitation signal B₁ is terminated, this signal may be received and processed to form an image.

When utilizing these signals to produce images, magnetic field gradients (Gₓ G_{y} and G_{z}) are employed. Typically, the region to be imaged is scanned by a sequence of measurement cycles in which an RF excitation pulse is applied and these gradients are varied according to a particular localization method. The resulting set of received NMR signals are digitized and processed to reconstruct the image using one of many well known reconstruction techniques. Such pulse sequences may also employ RF refocusing pulses, RF saturation pulses and other types of RF pulses required by the prescribed scan.

Very high field MR systems (such as MR scanners operating at a main field strength of 3.0 Tesla (T)) are becoming more widely available. An enabling technology is the compact, actively shielded magnets, which recently became available. This technology permits the 3.0 T MRI system to be sited in a clinical setting. Clinical applications including pulse sequences, and parameter selections (i.e. protocols) are being developed especially for these high field scanners.

A major limitation of scanning at very high field is the radiofrequency (RF) power deposited in the patient, as measured by the specific absorption rate (SAR). SAR increases approximately quadratically in the range of 1.5 T to 3.0 T. Therefore, applications which are straightforward to implement at standard fields strengths such as 1.5 T can be severely limited by SAR at higher field strengths such as 3.0 T. Specific guidelines for the maximal amount of SAR that may be deposited in the patient are specified by the Food and Drug Administration (FDA) in the United States, and by other regulatory agencies in other countries. If SAR limits are exceeded, undesirable and possible dangerous patient heating may result.

To ensure that SAR deposition is within acceptable limits, prior MR systems employ a number of measures. In one method, the RF power deposited by a particular pulse sequence is estimated with a calculation based on the shape, amplitude, and duration of each of the RF pulses within the pulse sequence. If the estimated SAR for a given pulse sequence exceeds regulatory limits, then the software automatically limits input parameters such as the maximal number of slices, flip angle, or minimal repetition time (TR).

Another method used in commercial MR systems employs power monitor hardware and software. The power monitor measures power transmitted by the RF coil in the MR system. In one commercial system, the average RF power delivered by the RF coil is measured at regular time intervals, approximately every 30 milliseconds (ms). A moving average of approximately 33 consecutive power measurements is calculated. Thus, the averaging time for this system is 30 ms×33 measurements, which is approximately 1 second. If at any time this moving average of measured power exceeds a predetermined limit (e.g. 10 Watts for head coil studies), the power monitor “trips”, and the scan is aborted. Such an RF power monitor method is disclosed in U.S. Pat. No. 6,426,623.

A limitation of such prior RF power monitoring methods is that they measure or predict the RF power that is delivered in bulk to the bore of the MRI system where the patient is positioned. These techniques assume that the RF power is evenly distributed throughout the volume and do not account for “hot spots” due to inhomogeneity of the applied RF field.

Thermal changes in substances undergoing MR imaging are known to cause spin resonance frequency shifts owing to changes in the magnetic susceptibility. As disclosed in U.S. Pat. Nos. 5,378,987; 5,711,300; and 6,377,834, this phenomenon is known as Proton Resonance Frequency (PRF) shift and it has been developed to produce temperature maps for use during interventional procedures in which tissues are heated with thermal ablation devices and the like. This method is used at medium and low field strengths (1.5 Tesla and below) and the temperature map provides an indication of tissue temperature in the region of interest being treated.

### SUMMARY OF THE INVENTION

The present invention is a method for determining the actual heating of tissues in a patient during a magnetic resonance imaging (MRI) scan and using that information to limit SAR exposure. In a prescan mode, a baseline thermal image is produced by acquiring image data from the subject with a pulse sequence that enables proton resonant frequency shifts due to temperature to be measured; a portion of the prescribed image acquisition pulse sequence is performed to produce RF heating in the subject; a second thermal image is acquired and produced to measure this heating; and the second thermal image is compared to the baseline thermal image to determine if an SAR violation will occur when using the prescribed imaging pulse sequence. Changes may be made in the prescribed imaging pulse sequence and the process repeated until the prescribed pulse sequence is set to optimal scan parameters. In a monitoring mode thermal images are periodically acquired during the actual acquisition of the prescribed image data; each thermal image is evaluated to determine if an SAR violation is occurring; and changes are made in the prescribed pulse sequence so that the scan can continue at an optimal rate without violating SAR rules.

A general object of the present invention is to provide a more accurate detection of SAR tissue heating and provide a more accurate control of the image acquisition process. The acquired thermal images may be examined on a pixel-by-pixel basis to actually measure tissue heating at specific locations in the subject. Hot spots may thus be found and used to control the image acquisition process rather than some average presumed temperature increase based on applied RF power.

Another object of the invention is to shorten the scan time of MRI procedures. Because prior SAR monitoring systems are based on RF power applied throughout the subject, very large safety factors are included to insure that tissue temperature does not increase to an undesirable level at any location in the subject. The present method actually measures tissue temperature increases throughout the subject and the rate of image data acquisition is slowed only when tissue temperature actually rises to an undesirable level at any location in the subject.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system which is operated to practice the present invention;

FIG. 2 is an electrical block diagram of the transceiver which forms part of the MRI system of FIG. 1;

FIG. 3 is a graphic representation of a preferred pulse sequence used to acquire thermal images with the MRI system of FIG. 1;

FIG. 4 is a flow chart of a method of using the present invention during a prescan mode of operating the MRI system of FIG. 1;

FIG. 5 is a flow chart of a method of using the present invention during a monitoring mode of operation; and

FIG. 6 is a graph indicating the correlation between thermal image phase and tissue temperature.

### GENERAL DESCRIPTION OF THE INVENTION

The PRF method for producing thermal maps has not been demonstrated to work at high field strengths. We hypothesized that at high field strengths the NMR signals acquired with a thermal imaging pulse sequence would have a higher signal to noise ratio (SNR), and that because the resonant frequency of spins is higher at high field strength, the phase shifts due to temperature change in tissue would be greater. In other words, at high field strengths which present a more difficult SAR problem, we can quickly acquire good and sensitive thermal images.

To test this hypothesis two identical gelatin phantoms were placed in the center of a GE Sigrna 3 T scanner (GE Medical Systems, Milwaukee, Wis.). Some T1 shortening Gd-DTPA contrast agent was added in the phantom to increase the SNR and in turn to improve temperature sensitivity of the measurement. One phantom was kept at room temperature to monitor non-thermal related system phase drift and the other phantom was heated to 45° C. and was cooled down during the course of 40 minutes. Three fiber optic thermal sensors (FISO FOT-L, Quebec, Canada) were embedded in each phantom to monitor the temperature change. The temperature reading was recorded at 2 second intervals and was synchronized to the MR image acquisition. After the mask image was acquired, the thermal images were acquired every 30 seconds with the following protocol: TR=7 ms, TE−3.22 ms, Flip Angle=30°, Bandwidth=±31.25 Khz, slice thickness=5.0 mm, acquisition matrix 256×256, FOV=32 cm, scan time 1.87s. For comparison, the same experiment was repeated in a GE Sigma 1.5 T scanner under similar conditions. The protocol was kept identical to that of the 3 T case with the exception of TE=3.30 ms.

Region of Interest (ROI) analysis was performed on the thermal phase images. A square region of 10 pixels in each dimension was selected at a location close to the thermal sensor and the average phases in that region was calculated. This value was corrected for the system background phase drift to yield a phase change that is only sensitive to the temperature change. A linear fit was applied to the phase change and thermal sensor data to obtain the phase-temperature.

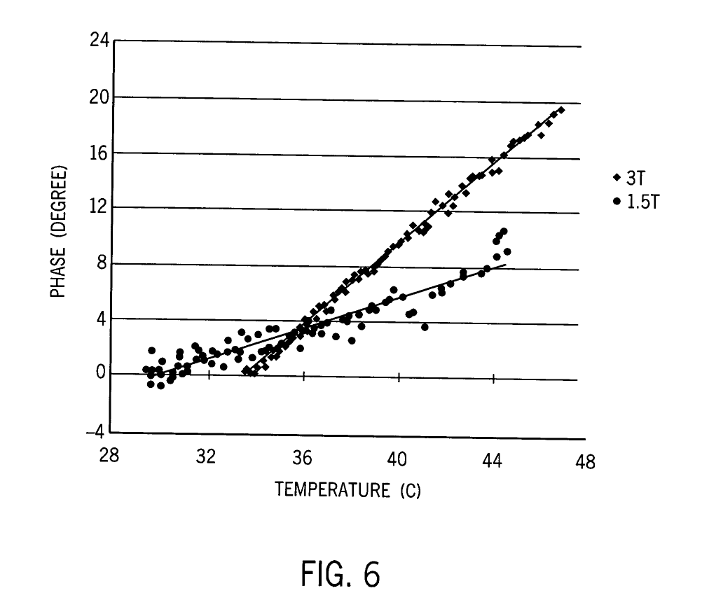

The SNR of the magnitude images are shown in column 1 in Table 1 and reflects a factor of 2 increase at a field strength of 3 T. This increased magnitude SNR results in better phase SNR in the phase different image, which is characterized inversely by the standard deviation in the phase images (column 2 in Table 1). The correlation between thermally induced phase change and the temperature measurements are higher at 3 T compared to 1.5 T as shown in FIG. 6. The correlation R is shown in column 3 in Table 1. The phase-temperature sensitivity is more than doubled at 3 T compared to 1.5 T. During the 40 minute scan, less than 1° system background phase drift was observed in the 3 T system while the similar drift was under 1.5° in the 1.5 T system.

TABLE 1

| | Magnitude | Phase | | Thermal Sensitivity |
|---|---|---|---|---|
| | SNR | STD (deg) | Correlation R | (deg/° C.) |
| 3 T | 210.0 | 2.66 | 0.9978 | 1.529 ± 0.010 |
| 1.5 T | 10.0 | 5.96 | 0.9464 | 0.580 ± 0.022 |

This demonstrates that the PRF shift based thermal imaging technique has increased sensitivity at 3 T and can be used in monitoring the RF heat deposition during a scan. This provides a new measurement tool to help build more realistic thermal models for prediction of SAR deposition, even on a patient-by-patent basis. It may be used during the imaging sequence to interactively monitor and control the scan. It can also be used as a pre-scan calibration to determine the optimal scan parameters without violating the safety limit. This allows further optimization of clinical protocols under 3 T and takes advantage of the higher field strength.

### DETAILED DESCRIPTION OF THE PREFERRED EMBODIMENT

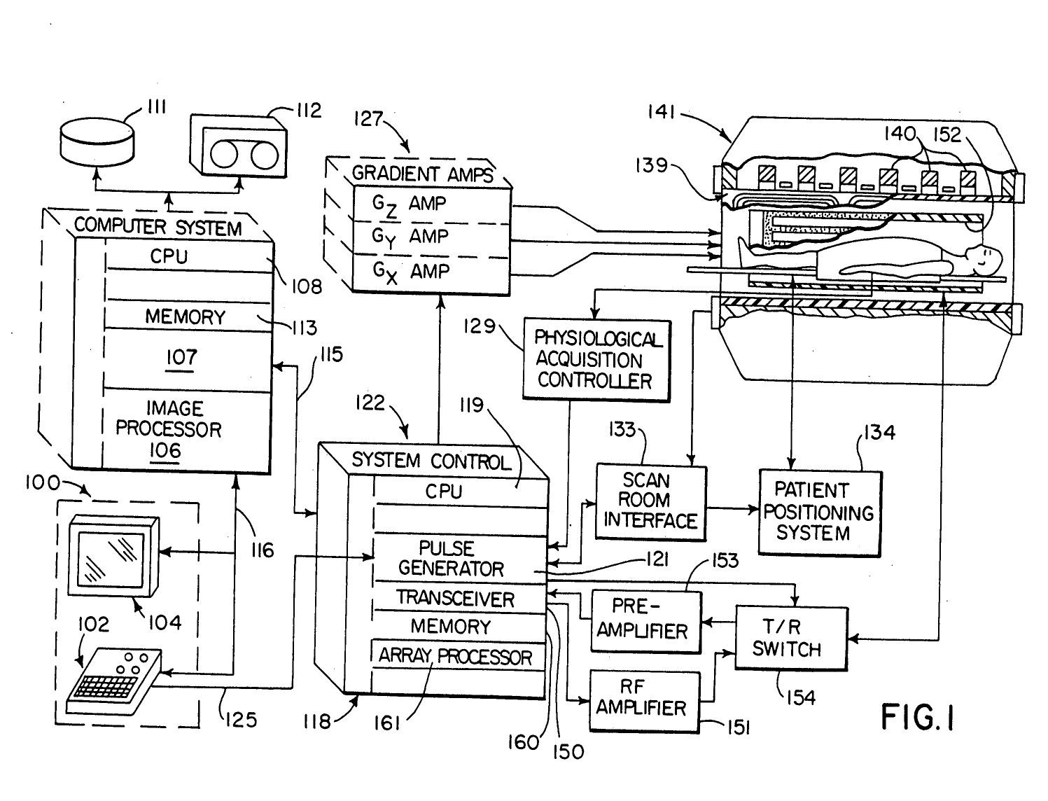

Referring first to FIG. 1, there is shown the major components of a preferred MRI system which incorporates the present invention. The operation of the system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules which communicate with each other through a backplane. These include an image processor module 106, a CPU module 108 and a memory module 113, known in the art as a frame buffer for storing image data arrays. The computer system 107 is linked to a disk storage 111 and a tape drive 112 for storage of image data and programs, and it communicates with a separate system control 112 through a high speed serial link 115.

The system control 122 includes a set of modules connected together by a backplane. These include a CPU module 119 and a pulse generator module 121 which connects to the operator console 100 through a serial link 125. It is through this link 125 that the system control 122 receives commands from the operator which indicate the scan sequence that is to be performed. The pulse generator module 121 operates the system components to carry out the desired scan sequence. It produces data which indicates the timing, strength and shape of the RF pulses which are to be produced, and the timing of and length of the data acquisition window. The pulse generator module 121 connects to a set of gradient amplifiers 127, to indicate the timing and shape of the gradient pulses to be produced during the scan. The pulse generator module 121 also receives patient data from a physiological acquisition controller 129 that receives signals from a number of different sensors connected to the patient, such as ECG signals from electrodes or respiratory signals from a bellows. And finally, the pulse generator module 121 connects to a scan room interface circuit 133 which receives signals from various sensors associated with the condition of the patient and the magnet system. It is also through the scan room interface circuit 133 that a patient positioning system 134 receives commands to move the patient to the desired position for the scan.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a whole-body RF coil 152. A transceiver module 150 in the system control 122 produces pulses which are amplified by an RF amplifier 151 and coupled to the RF coil 152 by a transmit/receive switch 154. The resulting signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate RF coil (for example, a head coil or surface coil) to be used in either the transmit or receive mode.

The NMR signals picked up by the RF coil 152 are digitized by the transceiver module 150 and transferred to a memory module 160 in the system control 122. When the scan is completed and an entire array of data has been acquired in the memory module 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 and conveyed to the operator console 100 and presented on the display 104.

Referring particularly to FIGS. 1 and 2, the transceiver 150 produces the RF excitation field B 1 through power amplifier 151 at a coil 152A and receives the resulting signal induced in a coil 152B. As indicated above, the coils 152A and B may be separate as shown in FIG. 2, or they may be a single wholebody coil as shown in FIG. 1. The base, or carrier, frequency of the RF excitation field is produced under control of a frequency synthesizer 200 which receives a set of digital signals from the CPU module 119 and pulse generator module 121. These digital signals indicate the frequency and phase of the RF carrier signal produced at an output 201. The commanded RF carrier is applied to a modulator and up converter 202 where its amplitude is modulated in response to a signal R(t) also received from the pulse generator module 121. The signal R(t) defines the envelope of the RF excitation pulse to be produced and is produced in the module 121 by sequentially reading out a series of stored digital values. These stored digital values may, in turn, be changed from the operator console 100 to enable any desired RF pulse envelope to be produced.

The magnitude of the RF excitation pulse produced at output 205 is attenuated by an exciter attenuator circuit 206 which receives a digital command, from the backplane 118. The attenuated RF excitation pulses are applied to the power amplifier 151 that drives the RF coil 152A. For a more detailed description of this portion of the transceiver 122, reference is made to U.S. Pat. No. 4,952,877 which is incorporated herein by reference.

Referring still to FIGS. 1 and 2 the signal produced by the subject is picked up by the receiver coil 152B and applied through the preamplifier 153 to the input of a receiver attenuator 207. The receiver attenuator 207 further amplifies the signal by an amount determined by a digital attenuation signal received from the backplane 118.

The received signal is at or around the Larmor frequency, and this high frequency signal is down converted in a two step process by a down converter 208 which first mixes the NMR signal with the carrier signal on line 201 and then mixes the resulting difference signal with the 2.5 MHz reference signal on line 204. The down converted NMR signal is applied to the input of an analog-to-digital (A/D) converter 209 which samples and digitizes the analog signal and applies it to a digital detector and signal processor 210 which produces 16-bit in-phase (I) values and 16-bit quadrature (Q) values corresponding to the received signal. The resulting stream of digitized I and Q values of the received signal are output through backplane 118 to the memory module 160 where they are employed to reconstruct an image.

The 2.5 MHz reference signal as well as the 250 kHz sampling signal and the 5, 10 and 60 MHz reference signals are produced by a reference frequency generator 203 from a common 20 MHz master clock signal. These provide a reference phase for the received NMR signals such that the phase is accurately reflected in the I and Q values. For a more detailed description of the receiver, reference is made to U.S. Pat. No. 4,992,736 which is incorporated herein by reference.

To practice the present invention a thermal image is acquired using an imaging pulse sequence, and an image is reconstructed in which the phase information at each image pixel is preserved. A two-dimensional image pulse sequence is employed in the preferred embodiment, and a two-dimensional Fourier transformation is performed on the acquired array of complex signal samples. The phase at each image pixel may be calculated as the argument of the complex value at the pixel: φ=—tan⁻¹Q/I. As will be described below, this phase measurement may be used to calculate a phase difference (Δφ) at each image pixel which indicates tissue temperatures. In the preferred embodiment a gradient recalled echo pulse sequence is employed to acquire this phase image data for the thermal image but other pulse sequences are also possible.

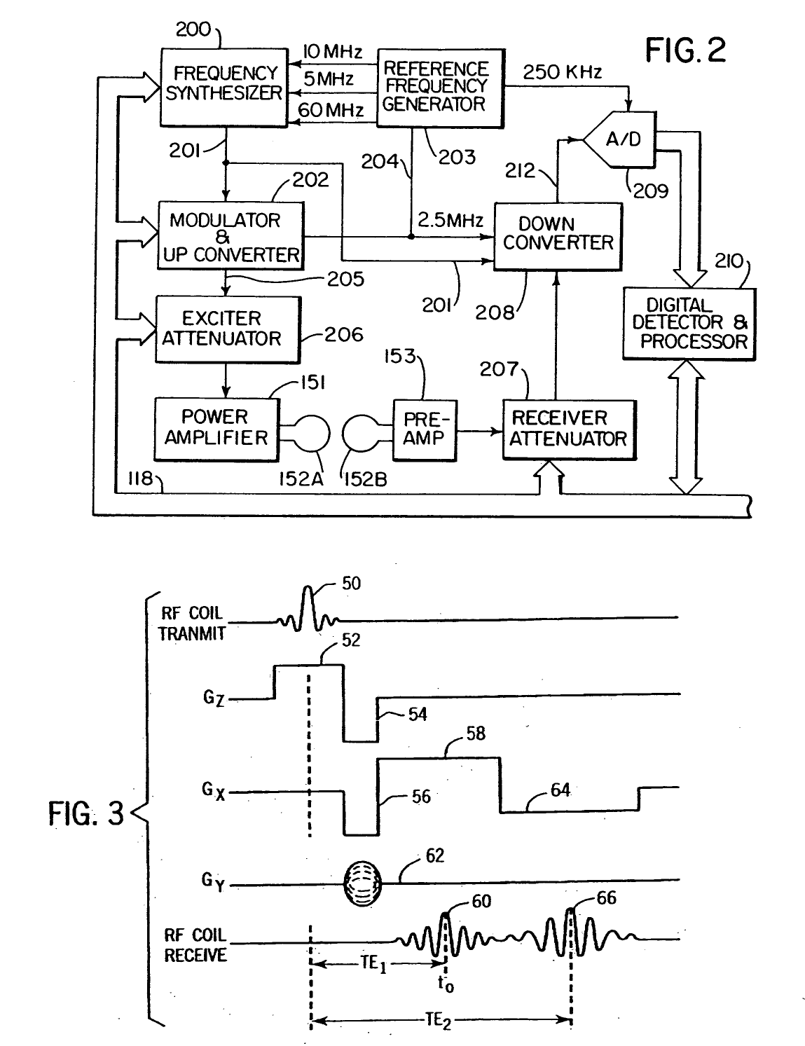

Referring to FIG. 3, a gradient echo pulse sequence begins with the transmission of a narrow bandwidth radio frequency (RF) pulse 50 in the presence of slice selection G_{z} pulse 52. The energy and the phase of this initial RF pulse may be controlled such that at its termination the magnetic moments of the individual nuclei are aligned in the x-y plane of a rotating reference frame of the nuclear spin system. A pulse of such energy and duration is termed a 90° RF pulse. The result of the combined RF signal and gradient pulse 52 is that the nuclear spins of a narrow slice in the three dimensional imaged object along spatial z-plane are excited. Only those spins with a Larmor frequency, under the combined field G_{z} and B₀, within the frequency bandwidth of the RF pulse will be excited. Hence the position of the slice may be controlled by the gradient G_{z} intensity and the RF frequency. A negative G_{z} rewinder gradient pulse 54, serves to rephase the nuclear spins in the x-y plane of the rotating frame. Rewinder pulse 54 therefore is approximately equal to half the area of that portion of slice select gradient 52 which occurs during the RF pulse 50.

After or during the application of the G_{z} rewinder pulse 54, the Gₓ prewinder pulse 56 is applied. Subsequently, a positive Gₓ readout pulse 58, centered at time TE₁, after the center of RF pulse 50 causes the dephased spins to rephase into a first gradient echo or NMR signal 60 at or near the center of the read-out pulse 58. The gradient echo 60 is the NMR signal for one row or column in a reference phase image. The read-out gradient Gₓ is then reversed to form a second read-out pulse 64, and a second gradient echo NMR signal 66 is formed and acquired. The second gradient echo 66 is centered at TE₂ and it produces the data for one row or column in a measurement image. As will become apparent below, the echo times TE₁ and TE₂ are selected very carefully to time the two echo signals 60 and 66 with the relative phase of fat and water spins. Variable bandwidth methods such as that described in U.S. Pat. No. 4,952,876 entitled. “Variable Bandwidth Multi-echo NMR Imaging” may also be used to advantage to improve SNR and is hereby incorporated by reference.

In a two dimensional imaging sequence, a gradient pulse G_{y} is applied to phase encode the spins along the y axis during the prewinder gradient 56. The sequence is then repeated with different G_{y} gradients, as is understood in the art, to acquire an NMR view set from which a tomographic image of the image object may be reconstructed according to conventional 2DFT reconstruction techniques.

The NMR signals 60 and 66 are the sum of the component signals from many precessing nuclei throughout the excited slice. Ideally, the phase of each component signal will be determined by the strength of the G_{z}, Gₓ and G_{y} gradients at the location of the individual nuclei during the readout pulses 58 and 64, and hence by the spatial z-axis, x-axis and y-axis locations of the nuclei. In practice, however, numerous other factors affect the phase of the NMR signals 60 and 66—including the temperature of the scanned tissues.

Tissue magnetic susceptibility changes as a function of temperature. This susceptibility change in turn causes spin resonance frequency shifts which vary linearly with temperature as shown in FIG. 6. A temperature map is produced according to the present invention by performing two phase image acquisitions. The first phase image is acquired with a short echo time (TE₁) selected from the above Table 3 with fat and water spins either in-phase or out-of-phase. This acquisition serves as a spatial, composition, relaxation time, and temperature reference phase image. A second phase image is acquired with an echo time (TE₂) selected from the above Table 3 with fat and water spins in the opposite condition to that of the reference acquisition. In other words, if the TE₁ for the reference phase image is chosen with fat and water spins in-phase, then the second phase image is acquired with fat and water spins out-of-phase, or visa versa. When the difference between the two phase images is used to produce a temperature map, the resulting temperature map is not affected by susceptibility and frequency changes due to the two types of spins present in the imaged tissues, and the accuracy of the measurement is substantially improved.

The information necessary to produce a temperature map is contained in the phase difference between the reference and measurement images. This information can be extracted in a number of ways. First, the phase difference (Δφ) may be calculated at each image pixel
Δφ=tan⁻¹Q₂/I₂−tan⁻¹Q₁/I₁.

These phase difference values (Δφ) are multiplied by a constant to produce numbers indicative of relative temperature. This is the preferred method when a quantitative temperature map is produced.

While a double echo pulse sequence is used in the above-described embodiment to acquire both phase images in a single scan, a single echo pulse sequence can also be used. In such case it is not necessary to repeat the reference image acquisition each time a temperature map is to be produced during a therapy procedure. If the first reference image is retained, subsequent phase images need only be acquired at the second echo time for self-referencing to be effective. However, if during the course of therapy significant tissue changes occur, it may be desirable to re-scan and update the reference phase image.

While a gradient-recalled echo pulse sequence is used to produce the phase images in the preferred embodiment, other well-known imaging pulse sequences can be used. Single and double spin echo pulse sequences can also be used, and either 2D or 3D pulse sequences will work. Also, while TE₁ and TE₂ are different in the preferred embodiment, this is not necessary. TE₁ and TE₂ can be the same. In this instance, TE₁ and TE₂ do not need to fall on fat-water in and out-of-phase boundaries, but may take on any value.

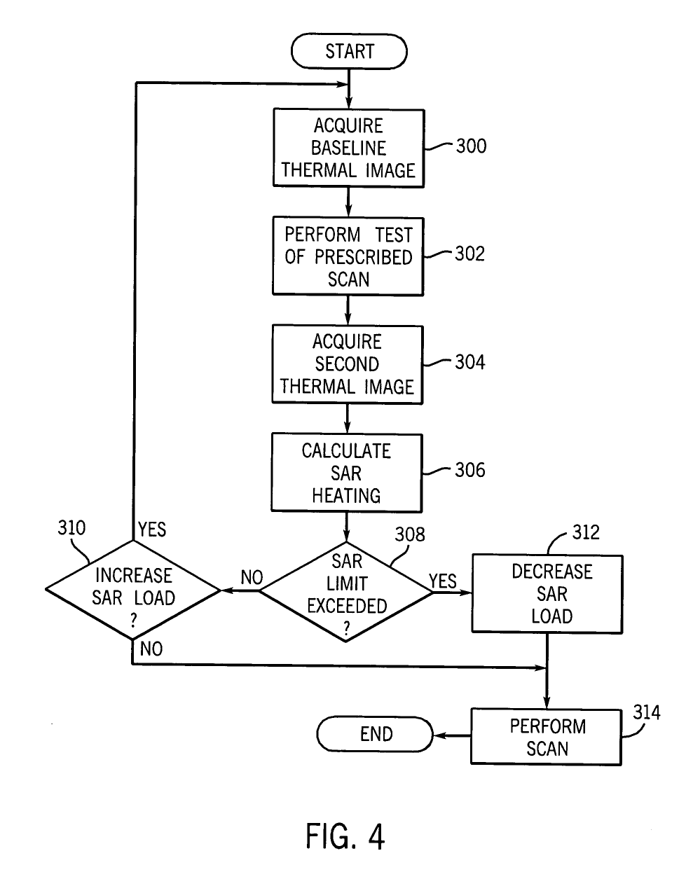

Referring particularly to FIG. 4, the present invention may be employed in a prescan mode to adjust the scan parameters of a prescribed image pulse sequence. This prescan method may be part of a larger prescan process that typically takes place at the beginning of an MRI scan. When started, the first step as indicated at process block 300 is to acquire a baseline thermal image using the above-described pulse sequence. This establishes tissue temperature before heating due to application of RF energy. This is followed by performing the prescribed imaging pulse sequence for a short time interval as indicated at process block 302. The important aspect of this step is to apply the prescribed RF pulses at the prescribed strength, or flip angle, and at the prescribed rate for a sufficient time interval to change tissue temperature. It is not necessary to apply phase encoding and readout gradient pulses during this step, although they can be applied.

As indicated at process block 304, the next step is to acquire a second thermal image using the above-described pulse sequence to determine the temperature of imaged tissues after the test step 302. As indicated at process block 306, the change in SAR temperature is then determined by subtracting the temperature at each pixel in the baseline thermal image from the corresponding pixel temperature in the second thermal image. The resulting difference image indicates the increase in tissue temperature due to the prescribed RF pulses.

As indicated at decision block 308, this difference image is examined to determine if the SAR limit is exceeded at any location therein. The magnitude of the phase change at each pixel is examined to determine if it exceeds a preset limit which indicates that excessive tissue heating is occurring at that location. If the SAR limit is not exceeded, a determination is made at decision block 310 whether the pulse sequence can be changed for the better with a resulting incremental increase in SAR load. If so, the pulse sequence is changed and the system loops back to repeat the prescan process. Otherwise, the optimal prescribed pulse sequence can be performed without exceeding the SAR limit and the prescan process is completed.

This process repeats until the SAR load has been increased to the point where the SAR limit has been exceeded as determined at decision block 308. When this occurs the SAR load produced by the prescribed sequence is reduced a preset amount as indicated at process block 312 and the prescan is completed. The imaging scan is then performed as indicated at process block 314.

It should be apparent to those skilled in the art that the SAR load produced by a prescribed pulse sequence can be increased or decreased in a number of ways. The flip angle of an RF pulse can be increased or decreased or the spacing between RF pulses can be changed. The preferred method will depend primarily on the type of pulse sequence being used, since RF pulse flip angle and pulse timing may or may not be a variable scan parameter. For example, in a fast spin echo pulse sequence the RF pulse flip angles must be set at 90° and 180°, although the spacing of 180° RF refocusing pulses can be easily changed to adjust SAR load.

The second embodiment of the invention is a monitoring mode of operation during the actual acquisition of MR image data. In this embodiment of the invention the scan is divided into segments and after each segment is acquired the SAR is checked to determine if the limit has been exceeded. Each scan segment is comprised of one or more prescribed pulse sequences with differing phase encodings or projection angles, which can cause a significant tissue temperature increase if the SAR limit is exceeded. For example, in a scan comprised of 256 repetitions of an 8 echo fast spin echo (FSE) pulse sequence, each segment may be 32 repetitions of the FSE pulse sequence.

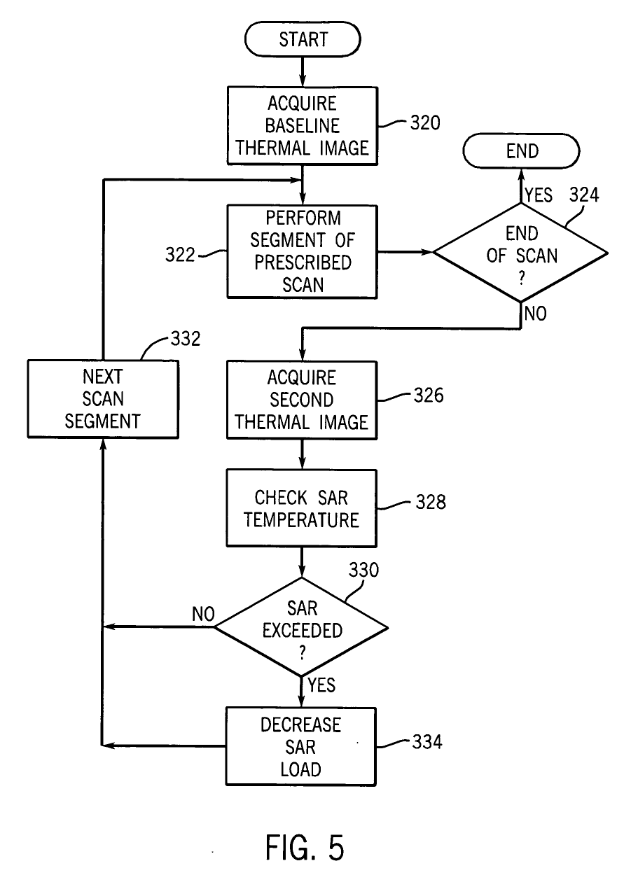

Referring particularly to FIG. 5, prior to the acquisition of a scan segment a baseline thermal image is acquired at process block 320. This employs the above described pulse sequence, but in order to minimize scan time, fewer phase encodings are acquired in this mode of operation. This results in a thermal image with lower spatial resolution, but a trade-off must be made between this and increased scan time. Following acquisition of the baseline thermal image, a segment of image data is acquired at process block 322 and a test is made at decision block 324 to determine if all segments have been acquired and the scan is complete.

After each segment is acquired during the scan a second thermal image is acquired at process block 326. This acquisition is identical to that used to acquire the baseline thermal image, and the pixel values in the baseline thermal image are subtracted from corresponding pixel values in the second thermal image at process block 328 to check the temperature increase at each pixel location. The temperature increase of each pixel location is checked at decision block 330, and if the SAR limit has not been exceeded, the system lops back to continue the scan of the next segment as indicated at process block 332. Note that in this embodiment it is not necessary to repeat the separate acquisition of the baseline thermal image because the second thermal image just acquired can serve as the baseline thermal image for the next iteration.

If the SAR limit is exceeded, however, the prescribed image pulse sequence is changed to reduce the SAR load as indicated at process block 334 before continuing the scan. This change can either be a reduction of RF pulse flip angle or a lengthening of interval between RF pulses as discussed above.

During the scan image data is acquired one segment at a time, and if at any point during the scan tissue temperature increases at any location within the field of view of the thermal image, the prescribed scan is automatically changed to reduce the SAR load on the subject. Incremental decreases in SAR load will be made until the excessive temperature increase is stopped.

It should be apparent that additional measures can also be taken when an excessive tissue temperature increase is detected. For example, if a second, higher preset temperature increase is detected the scan my be automatically terminated. Termination may also result if the prescribed pulse sequence is altered to such an extent at process block 334 that continued acquisition of image data is not reasonably feasible.

1. A method for detecting the absorption of RF energy by a subject during the acquisition of image data using a selected pulse sequence on a magnetic resonance imaging (MRI) system, the steps comprising:
a) acquiring a baseline thermal image of the subject using the MRI system;
b) performing the selected pulse sequence using the MRI system to expose the subject to RF energy produced by said pulse sequence;
c) acquiring a second thermal image of the subject using the MRI system; and
d) determining the temperature increase at locations in the subject using information in the baseline and second thermal images.

2. The method as recited in claim 1 in which step d) includes:
i) calculating a baseline phase image from the acquired baseline thermal image;
ii) calculating a phase image from the acquired second thermal image;
iii) subtracting values at locations in the baseline phase image from values at corresponding locations in the phase image.

3. A method for automatically altering a pulse sequence for acquiring image data from a subject on a magnetic resonance imaging (MRI) system, the steps comprising:
a) performing the pulse sequence using the MRI system to expose the subject to RF energy produced by said pulse sequence;
b) measuring the temperature increase produced at locations in the subject by acquiring a thermal image of the subject using the MRI system; and
c) altering the pulse sequence in response to the measured temperature increase to manage the SAR load on the subject.

4. The method as recited in claim 3 in which steps a), b) and c) are performed in a prescan procedure prior to the acquisition of image data from the subject using the pulse sequence.

5. The method as recited in claim 3 in which step a) acquires a segment of image data during the acquisition of image data from the subject, and steps a), b) and c) are repeated until all segments of image data have been acquired from the subject.

6. The method as recited in claim 3 in which step b) is performed by performing a pulse sequence on the MRI system which produces an NMR signal having a phase indicative of subject temperature.

7. The method as recited in claim 1 in which steps a) and c) include:
i) performing a pulse sequence with the MRI system to acquire NMR data from a region in the subject; and
ii) reconstructing an image from the acquired NMR data.

8. The method as recited in claim 7 in which step d) includes:
iii) calculating a baseline phase image from the reconstructed baseline thermal image;
iv) calculating a phase image from the reconstructed second thermal image; and
v) subtracting values at locations in the baseline phase image from values at corresponding locations in the phase image.

9. The method as recited in claim 7 in which the pulse sequence is a 2DFT pulse sequence and the reconstructed images are two-dimensional images of the region.

10. A method for operating a magnetic resonance imaging (MRI) system, the steps comprising:
a) acquiring a baseline thermal image of a subject to be scanned with the MRI system;
b) performing a pulse sequence using the MRI system to expose the subject to RF energy produced during the scan;
c) acquiring a second thermal image of the subject using the MRI system;
d) determining the temperature increase at locations in the subject using information in the baseline and second thermal images;
e) adjusting the pulse sequence to alter the temperature increase it produces in the subject; and
f) acquiring an image of the subject using an imaging pulse sequence in the MRI system which employs the adjusted pulse sequence.

11. The method as recited in claim 10 in which step d) includes:
i) calculating a baseline phase image from the acquired baseline thermal image;
ii) calculating a phase image from the acquired second thermal image;
iii) subtracting values at locations in the baseline phase image from values at corresponding locations in the phase image.

12. A method for scanning a subject with a magnetic resonance imaging (MRI) system, the steps comprising:
a) acquiring a baseline thermal image of the subject using the MRI system;
b) acquiring a plurality of NMR signals using an imaging pulse sequence, the NMR signals being used to produce an image of the subject;
c) acquiring a second thermal image of the subject using the MRI system;
d) determining the temperature increase at locations in the subject using information in the baseline and second thermal images;
e) repeating steps a) through d) until the image of the subject can be reconstructed from the acquired NMR signals; and
wherein the scan is altered if the temperature increase determined in step d) exceeds a predetermined amount.

13. The method as recited in claim 12 in which step d) includes:
i) calculating a baseline phase image from the acquired baseline thermal image;
ii) calculating a phase image from the acquired second thermal image;
iii) subtracting values at locations in the baseline phase image from values at corresponding locations in the phase image.

14. The method as recited in claim 12 wherein the scan is altered by stopping the scan.
